(19) United States
(12) Patent Application Publication
(10) Pub. No.: US 2013/0172979 A1
(43) Pub. Date: Jul. 4, 2013

(54) Medical Implant

(71) Applicant: BIOTRONIK AG, Buelach (CH)

(72) Inventor: Amir Fargahi, Buelach (CH)

(73) Assignee: BIOTRONIK AG, Buelach (CH)

(21) Appl. No.: 13/720,538

(22) Filed: Dec. 19, 2012

Related U.S. Application Data
(60) Provisional application No. 61/582,835, filed on Jan. 4, 2012.

(51) Int. Cl. A61F 2/962

(52) U.S. Cl. CPC A61F 2/962
USPC 623/1.12

(57) ABSTRACT

The invention is directed to a device (100) comprising a medical implant (30), in particular a stent, for implantation in an animal body and/or human body, wherein the medical implant (30) is disposed on a distal end (16) of a carrier (10) for positioning at the intended site, and the medical implant (30) is enclosed at least temporarily by an outer sleeve (20). The outer sleeve (20) on the distal end (18) of the carrier (10) is detachably attachable or attached to the carrier (10).

### CROSS REFERENCE TO RELATED APPLICATIONS

This application claims benefit of priority to U.S. patent application Ser. No. 61/582,835, filed Jan. 4, 2012; the contents of which are herein incorporated by reference in their entirety.

### TECHNICAL FIELD

The invention relates to a medical implant, in particular a stent, for implantation in an animal body and/or human body.

### BACKGROUND

Implants are used often in medical applications for implantation in an animal body and/or human body permanently or at least for an extended period of time to perform replacement functions. Examples would be e.g. cardiac pacemakers, brain pacemakers for Parkinson's patients, cardiac implants, cochlear implants, retinal implants, dental implants, joint replacement implants, vascular prostheses or stents.

It is known that stents that are connected to catheters can be released prematurely in the transport packaging thereof due to thermomechanical loading of the catheter. To prevent such a premature release, it is known to lock catheters in position in the proximal region which faces the user. In this case, however, the thermomechanical load acts in a concentrated manner on the distal region at which the stent is disposed, and therefore premature release can occur nevertheless.

Alternatively, the distance between the distal stent end and the end of the protective sleeve which protects the stent during transport and delivery to the intended location is also extended. The size of the distance is limited, however. The greater the distance is, the stiffer and less flexible the catheter becomes in the distal region. Furthermore, this makes it difficult to position the stent at the application site.

### SUMMARY

The problem addressed by the invention is that of providing a device including a medical implant, in the case of which the risk of premature release of the medical implant can be diminished.

The problem is solved according to the invention by the features discussed below. Favorable embodiments and advantages of the invention will become apparent from the drawings and the description.

The invention is directed to a device including a medical implant, in particular a stent, for is implantation in an animal body and/or human body, wherein the medical implant is disposed on a distal end of a carrier for positioning at the intended site, and the medical implant is enclosed at least temporarily by an outer sleeve.

The outer sleeve on the distal end of the carrier is detachably attachable or attached to the carrier.

Advantageously, the outer sleeve cannot become inadvertently detached from the carrier or the covered medical implant. A relative motion of the outer sleeve relative to the carrier is reliably prevented. Advantageously, the outer sleeve is attached or attachable such that the connection formed thereby on the distal end between the outer sleeve and the carrier withstands at least a tensile force exerted at the intended site when the medical implant is released. A typical tensile force for this purpose is a few newtons, e.g. 5 newtons. Unwanted separation of the outer sleeve and the carrier, in a transport packaging for instance, can be prevented in this manner.

“Attachable” is intended to mean, in particular, specially equipped, designed, and/or prepared.

In this context, an “implant” is intended to mean, in particular, a body that functions as a replacement, permanently or at least for a longer period of time, when implanted in an animal body and/or human body. Any medical implants that appear appropriate to a person skilled in the art would also be feasible here. A further example is a catheter including a self-expanding heart valve.

Furthermore, a “stent” in this context is intended to mean, in particular, a structure such as a wire mesh that substantially imparts a shape and/or form to the stent and/or forms the stent itself and is preferably actively expandable. In addition, the stent is preferably composed of an elastic or superelastic material such as a metallic material and/or a combination of a plurality of metallic materials, such as iron, magnesium, nickel, tungsten, titanium, zirconium, niobium, tantalum, zinc, silicon, lithium, sodium, potassium, calcium, manganese, and/or any other material that appears reasonable to a person skilled in the art.

The expression “actively expandable” is intended to mean, in particular, that the region is expanded or expandable automatically or autonomously, i.e. without outside assistance. Advantageously, the actively expandable region is made of a memory effect material, such as a copper-zinc-aluminum alloy and/or nickel-titanium alloy, preferably Nitinol. In addition, it would also be feasible to provide two actively expandable regions that have identical or different expansion characteristics. The expression “passively expandable” is intended to mean, in particular, that the region is expandable or plastically deformable non-autonomously and/or using an externally applied force. In particular, an expansion with the aid of a self-expanding material and/or by way of the first region is not implied here. The passive expansion can take place in a structurally simple manner using a balloon catheter.

Furthermore, it can be advantageous when the passively expandable region or the stent includes, at the least, cobalt and/or chromium, preferably in the form of stainless steel and/or medical stainless steel and/or a Cr—Ni—Fe steel—preferably the alloy 316L in this case—or a Co—Cr steel.

The expression “distal end of the carrier” is intended to mean, in particular, the end of the carrier to be inserted into the human or animal body to bring the medical implant to the intended site. The opposite, proximal end of the carrier faces the user and remains outside of the human or animal body.

The carrier can be a flexible shaft or tube which can contain, in the interior thereof, a guide wire, for example, or one or more lumina for gas or fluid. In particular, the carrier can be in the form of a catheter or an inner shaft of a catheter.

The expression “detachably attachable or attached to the carrier” is intended to mean, in particular, that separation from the outer sleeve and carrier can take place preferably without the use of tools and, in particular, that damage to the outer sleeve and/or carrier is ruled out under normal conditions when the outer sleeve is detached from the carrier.

Advantageously, the outer sleeve can be connected in a non-positive or form-fit manner to a distal region of the carrier. This makes it possible to axially secure the outer sleeve on the carrier.

According to a favorable embodiment, the outer sleeve can include, in the distal region, at least one bore transverse to a longitudinal axis of the carrier, which aligns with a bore in the carrier. Advantageously, a connecting element is routed or routable through the at least one bore in the outer sleeve and the at least one bore in the carrier. Preferably the connecting element can be a wire piece.

Favorably, the wire piece can have a diameter that is smaller than the diameter of a guide wire which can be provided in the carrier to deliver the medical implant to the intended site. “Provided” is intended to mean, in particular, specially equipped, designed, and/or prepared.

For example, the diameter of the wire piece can be approximately half as thick as the guide wire. The material of the wire piece can be metallic or non-metallic. For example, the wire piece can be made of stainless steel or Nitinol, or a plastic such as polyamide (PA), polyaramide (e.g. Kevlar from DuPont), polyethylene (PE), polyethylene terephthalate (PET) and the like.

Alternatively or additionally, the outer sleeve is clamped or clampable in the distal region on the carrier.

By “clamped” or “clampable”, it is meant that the outer sleeve is pressed or pressable onto the carrier in the radial direction. This can take place circumferentially around the circumference of the outer sleeve or, for example, at two or more regions of the outer sleeve, which are opposite one another on the carrier. “Attachable” is intended to mean, in particular, specially equipped, designed, and/or prepared.

The outer sleeve can be clamped or clampable to the carrier in the distal region using a clamping device. The clamping device can be in the form of an elastic band, for instance, or in the form of a tube clamp, or in the form of a cording. The type of clamping device can be matched to the special application or the particular medical implant.

Advantageously, the carrier can have a constriction, in the region of which the outer sleeve is clamped or clampable. The clamped region of the outer sleeve can therefore be secured axially to the carrier. Available constrictions on the carrier can also be used, and therefore practically nothing about the carrier need be changed.

### DESCRIPTION OF THE DRAWINGS

The invention is explained in the following in greater detail with reference to embodiments that are depicted in drawings. They show, in a diagrammatic representation:

FIG. 1 a view of a self-expanding medical implant on a carrier, which has been released from the outer sleeve thereof;

FIG. 2 a first embodiment of a device according to the invention, including a securing wire; and

FIG. 3 a further embodiment of a device according to the invention, including a clamping device.

### DETAILED DESCRIPTION

Elements that are functionally identical or similar-acting are labelled using the same reference symbols in the figures. The figures are schematic depictions of the invention. They do not depict specific parameters of the invention. Furthermore, the figures merely show typical embodiments of the invention and are not intended to limit the invention to the embodiments shown.

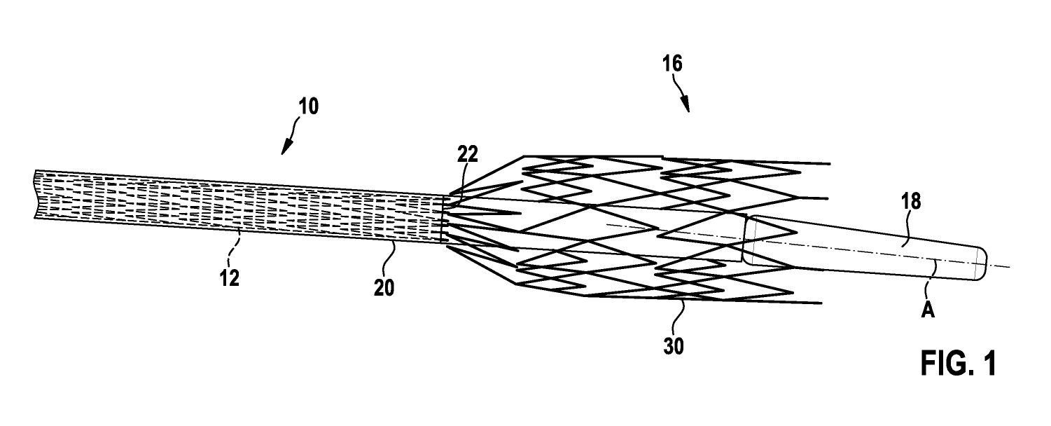

FIG. 1 shows an example of a self-expanding medical implant 10 in the form of a self-expanding stent on a carrier 12 in the form of an inner shaft of a catheter including a catheter tip 18, wherein the medical implant 10 was unintentionally released from the outer sleeve 20 thereof by thermomechanical load since the outer sleeve 20 has slid axially, via terminal edge 22 thereof, underneath the position of the medical implant 10. The medical implant 30 has become detached from the crimped position thereof on the carrier 12, in which the medical implant 30 should be brought to the intended site, and has expanded. The medical implant 30 must therefore be discarded.

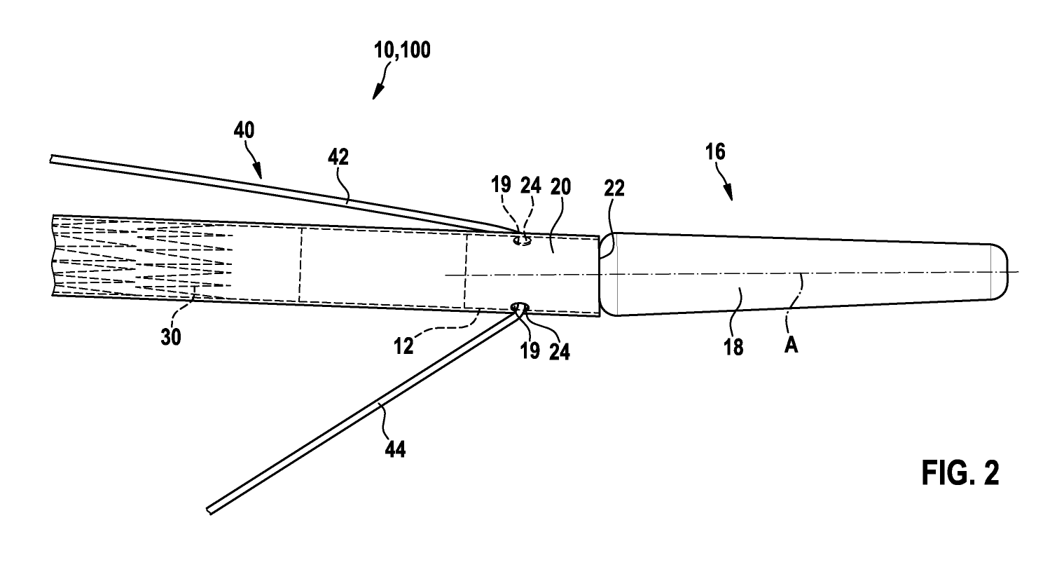

FIG. 2 shows an embodiment of a device 100 according to the invention, including a medical implant 30 in the form of a stent, for implantation in an animal and/or human body. The medical implant 30 is disposed on a distal end 16 of a carrier 10 in the form of an inner shaft 12 of a catheter for positioning at the intended site. The catheter includes, on the rigid catheter tip 18 thereof, a longitudinal axis A, while the inner shaft 12 is designed to be largely flexible.

The medical implant 30 is enclosed in an outer sleeve 20 for transport and delivery to the intended site. The outer sleeve 20 is detachably attached to the carrier 10 on the distal end 16 of the carrier 10.

In the embodiment according to FIG. 2, the outer sleeve 20 is connected in the distal region 16 to the inner shaft 12 by a connecting means 40 which is routed through corresponding bores 24 in the outer sleeve 20 underneath the terminal end 22 thereof and bores 19 in the inner shaft transversely to the longitudinal axis A of the carrier 10. The connecting means 40 is in the form of a wire piece in this case, which extends toward both sides via an end 42, 44. The outer sleeve 20 is now secured against axial motion relative to the inner shaft 12.

The wire piece (connecting means 40) can have a very small diameter which can be in the range between 100 μm and 200 μm. In particular, the diameter is less than that of a typical guide wire of the type provided in catheters, the diameter of which is typically more than 400 μm. The bores 24, 19 are advantageously slightly larger than the diameter of the wire piece (connecting means 40) in order to ensure easy insertion of the wire piece 40.

The ends 42, 44 of the wire piece (connecting means 40) can be linked to one another using an easily loosened knot or by twisting the ends 42, 44, to secure the connection. The wire piece can be easily removed by the user.

Since the connecting means 40 is routed through the outer sleeve 20 and the inner shaft 12, it must be removed before the guide wire can be inserted into the catheter.

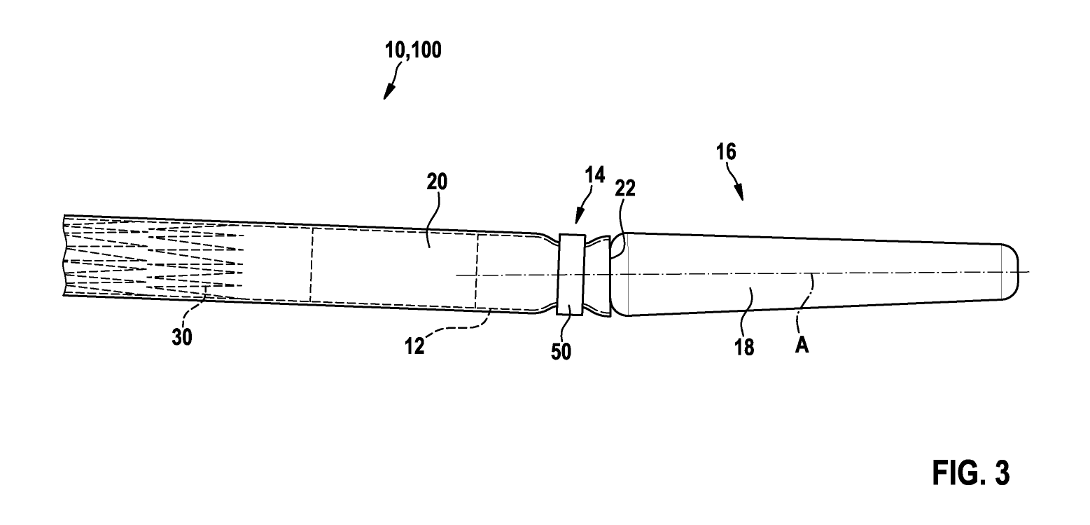

In an alternative embodiment according to FIG. 3, the outer sleeve 20 is clamped in the distal region 12 on the carrier 10. The basic design of the device 100 corresponds largely to that of the device in FIG. 2, and therefore reference is made to the figure description for FIG. 2 to avoid unnecessary repetition.

The connecting means 40 shown in FIG. 2 is replaced in this case by a clamping device 50, using which the outer sleeve 20 in the distal region 16 is clamped on the carrier 10. The carrier 10 or the inner shaft 12 includes a constriction 14, in the region of which the clamping device 50 can be disposed.

The clamping device 50 can be a wire loop or a tube clamp or a bandage.

A clamping device as a type of clamp is also feasible, which extends beyond the catheter tip 18 and clamps the outer sleeve 20 on the inner shaft 12.

A few examples of materials which can be used for the outer sleeve 20 and the inner shaft (carrier 10) having a so-called monolayer design are summarized below. The outer sleeve 20, can use at least one material such as ABS (acrylonitrile butadiene styrene), ANS (acrylonitrile styrene), Delrin acetal (an unfilled homopolymer, e.g. from DuPont), PVC (polyvinyl chloride), PEN (polyethylene naphtalate), PBT (polybutylene terephthalate), PC (polycarbonate), PEEK (polyetheretherketone), PEI (polyetherimide), PES (polyethersulfone), PET (polyethylene terephthalate), PETG (polyethylene terephthalate glycol), PA (polyamide), polyether, polyester, Kynar ADX (a thermoplastic fluorinated functionalized polymer from Arkema), PEBAXs (all hardnesses) a polyether block amide from Arkema), and PTFE (polytetrafluorethylene). The carrier 10 can use at least one material such as PI (polyimide); Hytrel (a polyester elastomer block copolymer from DuPont); PMMA (polymethylmethacrylate); PUR (polyurethane); EVA (ethylene vinyl acetate); ethylene vinyl alcohol embedded in PE (low, linear low, medium, high density polyethylene); latex rubber; FEP (perfluoro (ethylene propylene) plastic); TFE (tetrafluorethylene); PFA (perfluoroalkoxyl alkane); polypropylene; polysiloxane; and liquid crystal polymers such as silicon rubber, SAN (styrene acrylonitrile) and PA (polyamide), in particular PA-6, -6.6, -6.10, -6.12, -11, -12).

A few examples of materials which can be used for the outer sleeve 20 and the inner shaft (carrier 10) having a so-called multilayer-coextruded design are summarized below. The outer layer can use at least one material such as PI (polyimide), PEBAXs (all hardnesses) a polyether block amide from Arkema); and PA (polyamide), in particular PA-6, -6.6, -6.10, -6.12, -11, -12). The middle layer can use materials that are suitable as primers, i.e. that promote adhesion between the outer layer and the inner layer, such as linear low density polyethylene. The inner layer can use at least one material such as FEP (perfluoro (ethylene propylene) plastic); HDPE (high density polyethylene); and PTFE (polytetrafluorethylene) or Teflon.

Favorably, such a catheter having a so-called multilayer-coextruded design can be designed such that the outer sleeve and the inner shaft (carrier 10) each include an outer layer, a middle layer, and an inner layer.

A few examples of materials which can be used for the outer sleeve 20 and the inner shaft (carrier 10) having a so-called multilayer-coiled/braided-reinforced design are summarized below. The outer layer can use at least one material such as PI (polyimide); PEBAXs (all hardnesses); and PA (polyamide), in particular PA-6, -6.6, -6.10, -6.12, -11, -12). The braid/coil layer can use at least one material such as Nitinol wire (e.g. round wire, flat wire), stainless steel wire (e.g. round wire, flat wire), and polyaramid fiber, such as Kevlar from DuPont. The inner layer can use at least one material such as FEP (perfluoro (ethylene propylene) plastic), HDPE (high density PE), and PTFE (polytetrafluorethylene) or Teflon.

Favorably, such a catheter having a so-called multilayer-coiled/braided reinforced design can be designed, for example, such that the outer sleeve and the inner shaft (carrier 10) each include an outer layer, a middle layer, and an inner layer. In a braid-reinforced design, the middle layer is reinforced using a braid of a metallic fiber or plastic fiber integrated into the middle layer, or carbon-fiber reinforced, or the middle layer is made of such a braid. In a coil-reinforced design, the middle layer is reinforced with a coil integrated in the middle layer, which can include a metallic wire having any cross section (e.g. round wire, flat wire, etc. or any combination thereof), or plastic fiber or carbon fiber which are wound with one or more windings to form a coil, or the middle layer is composed of such a coil. The middle layer can be reinforced with mesh and with a coil.

A few examples of materials which can be used for the outer sleeve 20 and the inner shaft (carrier 10) having a so-called multilayer-braided-reinforced design are summarized below. The outer layer can use at least one material such as PI (polyimide); PEBAXs (all hardnesses); and PA (polyamide), in particular PA-6, -6.6, -6.10, -6.12, -11, -12). The braid layer can use at least one material such as Nitinol wire (having any cross section, e.g. round wire, flat wire etc., or any combination thereof), stainless steel wire (having any cross section, e.g. round wire, flat wire etc., or any combination thereof), cobalt-chromium alloy wire (having any cross section, e.g. round wire, flat wire etc., or any combination thereof), and polyaramid fiber (e.g. Kevlar from DuPont). The inner layer can use at least one material such as FEP (perfluoro (ethylene propylene) plastic), HDPE (high density PE), and PTFE (polytetrafluorethylene) or Teflon

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A device comprising a medical implant, optionally a stent, for implantation in an animal body and/or human body, wherein the medical implant is disposed on a distal region of a carrier for positioning at an intended site, and the medical implant is enclosed at least temporarily by an outer sleeve, characterized in that the outer sleeve is detachably attached to the carrier at the distal region of the carrier.

2. The device according to claim 1, characterized in that the outer sleeve is connectable or connected to the distal region of the carrier in a non-positive manner.

3. The device according to claim 1, characterized in that the outer sleeve is connectable or connected to the distal region of the carrier in a form-fit manner.

4. The device according to claim 1, characterized in that the outer sleeve comprises, in the distal region, at least one bore transverse to a longitudinal axis of the carrier, which aligns with a bore in the carrier.

5. The device according to claim 4, characterized in that a connecting element is routable or routed through the at least one bore in the outer sleeve and the at least one bore in the carrier.

6. The device according to claim 5, characterized in that the connecting element is a wire.

7. The device according to claim 6, characterized in that the wire has a diameter that is less than the diameter of a guide wire provided in the carrier to deliver the medical implant to the intended site.

8. The device according to claim 1, characterized in that the outer sleeve is clamped or clampable in the distal region to the carrier.

9. The device according to claim 8, characterized in that the outer sleeve is clamped or clampable in the distal region to the carrier using a clamping device.

10. The device according to claim 8, characterized in that the carrier has a constriction, in the region of which the outer sleeve is clamped or clampable to the carrier.
